(19) United States
(12) Plant Patent Application Publication
(10) Pub. No.: US 2002/0194659 P1
(43) Pub. Date: Dec. 19, 2002

(54) Forsythia plant named "Courtalyn"

(76) Inventor: M. Alain Cadic, Beaucouze (FR)

Correspondence Address: Benton S. Duffett, Jr., BURNS, DOANE, SWECKER & MATHIS, L.L.P., P.O. Box 1404, Alexandria, VA, 22313-1404, US

(21) Appl. No.: 10/217,519

(22) Filed: Aug. 14, 2002

Related U.S. Application Data
(63) Continuation of application No. 09/322,161, filed on May 28, 1999.

(51) Int. Cl. A01H 5/00

(52) U.S. Cl. PLT/230

(57) ABSTRACT

A new and distinct cultivar of Forsythia plant is provided that is an induced mutation of the ‘Lynwood’ cultivar (non-patented in the United States). The gamma radiation of buds was utilized to create the mutation. The new cultivar can be readily distinguished from the parent cultivar by the presence of a more uniform and compact upright growth habit which yields a plant approximately two-thirds the height of the parent cultivar. Showy golden yellow blossoms are presented over the entire plant in the springtime prior to the appearance of the leaves. The winter hardiness is good. The new cultivar is particularly well suited for growing as attractive ornamentation in the landscape.

### SUMMARY OF THE INVENTION

A new and distinct variety of Forsythia×intermedia plant is provided that is a mutation of the ‘Lynwood’ cultivar (non-patented in the United States).

The parent ‘Lynwood’ cultivar is a branch sport of the ‘Spectabilis’ cultivar that originally was found in garden in Cookstown, County Tyrone, Ireland.

When creating the new cultivar of the present invention during the 1970's, buds on one year-old stems of the ‘Lynwood’ cultivar were irradiated with gamma rays of cobalt 60 and subsequently were observed. Such irradiation was carried out at the Institut National De La Recherche Agronomique located at Angers, France. The irradiated plants were isolated and were kept for one month in the winter in a cold room.

It was found that a single branch on a single plant following such irradiation possessed the characteristics of the Forsythia cultivar of the present invention. The characteristics of such new cultivar can be summarized as follows:

(a) forms a more uniform and regular upright growth habit than the parent ‘Lynwood’ cultivar,

(b) exhibits a more compact growth habit than the parent ‘Lynwood’ cultivar with shorter internode lengths and forms a plant that is approximately two-thirds the height of the ‘Lynwood’ cultivar,

(c) forms attractive golden yellow blossoms over the entire plant in the springtime,

(d) propagates well by the use of softwood cuttings,

(e) is well suited for growing in containers, and

(f) exhibits good winter hardiness.

The new cultivar of the present invention well meets the needs of the horticultural industry and is particularly well suited for growing as attractive ornamentation in the landscape. Showy golden yellow blossoms are formed in abundance in the springtime before the leaves appear. The blossom configuration and color are the same as those of the ‘Lynwood’ cultivar. The growth habit is graceful and is more uniformly appearing than that of the parent ‘Lynwood’ cultivar. The new cultivar forms more laterals and the shoots are more numerous giving the plant a fuller appearance. Fewer wild and non-uniform branches are formed. The leaves are substantially the same as those of the ‘Lynwood’ cultivar.

The new cultivar has been found to readily undergo asexual propagation by the use of softwood cuttings. Roots readily form on such cuttings during the summer. Such asexual propagation has been carried out at Angers, France, and elsewhere.

The new cultivar has been named the ‘Courtalyn’ cultivar and is being marketed under the WEEK-END trademark.

### BRIEF DESCRIPTION OF THE PHOTOGRAPH

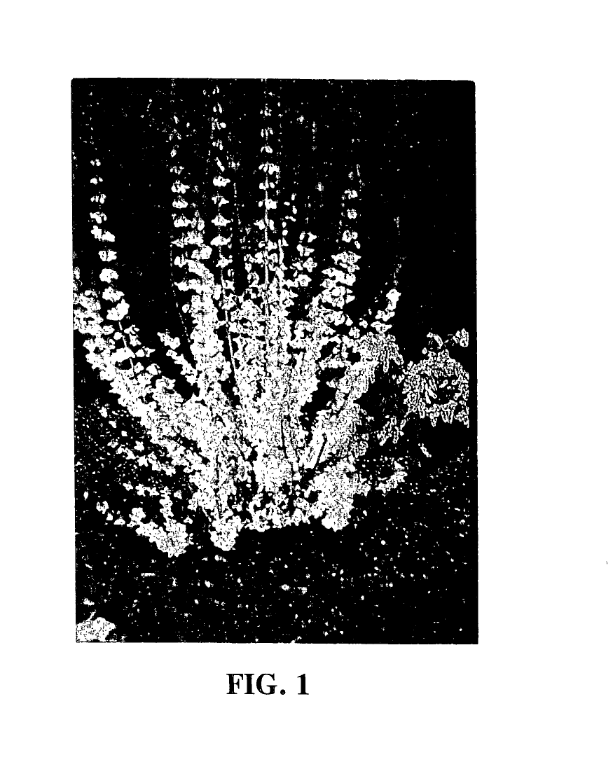

The accompanying FIG. 1 shows a typical plant of the new variety when blossoming during the springtime. The plant was approximately two to three years of age and was being grown outside at Angers, France. The relatively uniform disposition of the generally erect and upright branches is apparent as is the profuse display of blossoms over the entire plant.

### DETAILED DESCRIPTION

The following description is based on the observation of plants of the new cultivar growing outdoors at Angers, France. Such plants had been asexually reproduced through the use of softwood cuttings. The color terminology utilized in the description that follows is to be accorded its ordinary dictionary significance.

ORIGIN: Induced mutation. Buds present on one year-old stems were irradiated with gamma rays derived from cobalt 60.

PARENTAGE: ‘Lynwood’ cultivar (non-patented in the United States).

CLASSIFICATION: Forsythia×intermetia, cv. ‘Lynwood’.

PLANT:

form.—well-branched flowering shrub.

habit.—upright, graceful, and compact. The plant has a fuller more uniform appearance with fewer wild branches than the parent. ‘Lynwood’ cultivar. internode

length.—shorter than that of the parent ‘Lynwood’ cultivar.

height.—approximately two-thirds that of the parent ‘Lynwood’ cultivar. A twelve year-old plant of the ‘Courtalyn’ cultivar that has been trimmed regularly commonly will exhibit a height of approximately 1.5 meters.

width.—a twelve year-old plant of the ‘Courtalyn’ cultivar that has been trimmed regularly commonly will exhibit a width of approximately 1 meter.

FOLIAGE:

disposition.—opposite.

configuration.—substantially the same as that of the parent ‘Lynwood’ cultivar.

color.—substantially the same as that of the parent ‘Lynwood’ cultivar.

INFORESENCE:

configuration.—substantially the same as the parent ‘Lynwood’ cultivar.

size.—substantially the same as the parent ‘Lynwood’ cultivar.

color.—showy, golden yellow and substantially the same as the parent ‘Lynwood’ cultivar.

fragrance.—none.

disposition.—spread substantially evenly throughout the plant (as illustrated). time of

flowering.—prior to formation of leaves. During 1990 to 1993 commonly bloomed for 15 to 25 days at Angers, France. The earliest blooming date was February 25th and the latest blooming date was April 10th. This compares to a blooming duration of 10 to 20 days for the parent ‘Lynwood’ cultivar. Also, the earliest blooming date was February 20th and the latest blooming date was March 30th for the ‘Lynwood’ cultivar. Often the new cultivar flowers slightly earlier than the ‘Lynwood’ cultivar.

fruit bearing.—none.

GROWING CONDITIONS: Does well in full sun, and prefers soil that is not exceedingly dry.

DISEASE RESISTANCE: Substantially the same as that of the parent ‘Lynwood’ cultivar and not particularly susceptible to diseases. The slight presence of Pseudomonas and Botrytis may be observed in the springtime following blooming.

HARDINESS: Very good as is the parent ‘Lynwood’ cultivar. Has withstood temperatures of −18° C. at Angers, France.

PROPAGATION: Can be readily propagated while utilizing softwood cuttings.

USAGE: Attractive erect ornamental spring-flowering shrub for the landscape that requires little care.

I claim:

1. A new and distinct Forsythia cultivar that is a mutation of the ‘Lynwood’ cultivar having the following combination of characteristics:
(a) forms a more uniform and regular growth habit than the parent ‘Lynwood’ cultivar,
(b) exhibits a more compact growth habit than the parent ‘Lynwood’ cultivar with shorter internode lengths and forms a plant that is approximately two-thirds the height of the ‘Lynwood’ cultivar,
(c) forms attractive golden yellow blossoms over the entire plant in the springtime,
(d) propagates well by the use of softwood cuttings,
(e) is well suited for growing in containers, and
(f) exhibits good winter hardiness; substantially as illustrated and described.
